(19) United States
(12) Patent Application Publication
(10) Pub. No.: US 2021/0259949 A1
(43) Pub. Date: Aug. 26, 2021

(54) USE OF A BIXA ORELLANA EXTRACT

(71) Applicant: BASF Beauty Care Solutions France SAS, Lyon (FR)

(72) Inventors: Nicolas BERTHELEMY, Pagny sur Moselle (FR); Louis DANOUX, Saulxures-les-Nancy (FR); Philippe MOSER, Dommartemont (FR); Sabine PAIN, Vienne (FR); Nicolas PELLETIER, Lyon (FR)

(21) Appl. No.: 17/251,247

(22) PCT Filed: Jun. 6, 2019
(86) PCT No.: PCT/FR2019/051350

§ 371 Date: Dec. 11, 2020

(30) Foreign Application Priority Data
Jun. 11, 2018 (FR) 1855079

(51) Int. Cl. A61K 8/9789; A61Q 19/00; A61Q 5/00; A61K 36/185; A61K 8/02; A61K 8/73; A61P 17/08

(52) U.S. Cl. CPC A61K 8/9789; A61Q 19/008; A61Q 5/008; A61P 17/08; A61K 8/0275; A61K 8/732; A61K 36/185

(57) ABSTRACT

The present invention relates to the cosmetic use of a Bixa orellana extract for preventing and/or reducing sebum secretion and/or for preventing and/or reducing the unaesthetic and/or unpleasant and/or uncomfortable manifestations of sebum secretion. It also relates to a cosmetic care method, characterized in that it comprises the topical application to at least one area of skin, advantageously of the scalp, of a Bixa orellana extract for preventing and/or reducing sebum secretion and/or for preventing and/or reducing the unaesthetic and/or unpleasant and/or uncomfortable manifestations of sebum secretion. Finally, it relates to a Bixa orellana extract for use in the treatment and/or prevention of a pathological condition associated with sebum hyperproduction chosen from pathological hyperseborrhoea and seborrhoeic eczema and/or any combination thereof.

The present invention relates to the use of a Bixa orellana extract for preventing and/or reducing sebum secretion and/or the unaesthetic, unpleasant and/or uncomfortable manifestations linked to sebum secretion, in particular in cosmetic or pharmaceutical, in particular dermatological, compositions.

Sebum is produced by the sebaceous glands of the skin and consists of a complex mixture of lipids, in particular triglycerides (30-50%), free fatty acids (15-30%), wax esters (26-30%) and squalene (12-20%).

The sebaceous glands are located in the skin where, in combination with the hair follicle, they form the pilosebaceous unit. They are more numerous on the scalp and the face, reaching up to 400-900 glands/cm². The principal function of the sebaceous glands is sebum secretion from mature sebocytes. The secretion is of holocrine type, that is to say that when excreting the sebum in the follicular duct, the sebocytes die. The peripheral sebocytes proliferate and then differentiate into mature sebocytes at the centre of the gland, the latter being characterized by a high density of cytoplasmic lipid droplets. Complete sebocyte renewal in the sebaceous glands lasts approximately 4 weeks.

Sebum production is essential for good skin condition and function. Sebum is intended in particular to protect the skin from drying out, and from microbes by acidification, and to preserve its suppleness. Antibacterial and pheromone transport roles have been attributed to the sebum, and also an antioxidant role in particular linked to vitamin E production.

Too low a sebum production level thus results in the occurrence of dry, more fragile skin and in the loss of hair follicle function that can result in hair loss.

While sebum secretion is useful by virtue of these protective functions on the skin, when it is excessive, it confers imperfections on the skin, such as for example an appearance that is often described as shiny, thick, oily, pore dilation and/or blackhead formation, and hyperkeratinization of the skin, all of which are manifestations that are often perceived as unaesthetic and/or unpleasant and/or uncomfortable. These manifestations characterize skin referred to as “oily skin”.

The amount and the quality of sebum secreted varies according to skin type, for example Caucasian, Asian and African; however, the oily skin phenomenon is thought to affect 80% of individuals worldwide and thus constitutes a real multi-ethnic cosmetic problem.

In addition, this seborrhoea can be responsible for actual pathological conditions, such as seborrhoeic eczema, and/or hypersecretion in newborn infants (commonly denoted “cradle cap”).

While the principal origin of oily skin is sebocyte hyperactivity, with excessive sebum secretion and excretion by the sebaceous glands, multiple intrinsic factors (age, hormones, stress, etc.) and/or extrinsic factors (temperature, humidity, aggressive agents such as pollutants, etc.) influence the quality and/or amount of sebum secreted. Thus, the androgenic hormones and the excess growth factors produced during puberty are the principal factors involved in the development of oily skin. Moreover, an excess of the growth factor insulin-like growth factor type 1 (IGF1) is associated, on the one hand, with sebocyte hyperproliferation resulting in sebum overproduction and, on the other hand, with keratinocyte hyperproliferation and differentiation resulting in hyperkeratinization and therefore thickened skin.

There are numerous active agents in the cosmetic and dermatological fields for preventing and/or reducing sebum secretion directly. These “sebum-regulating”agents, such as for example zinc gluconate and azelaic acid, sarcosine and/or an Orthosiphon stamineus extract, directly reduce sebum production by the sebaceous glands. Other agents are also used for their indirect action on sebum secretion, in particular exfoliating, cell renewal-stimulating, astringent and/or matifying agents, by sebum absorption.

However, there is a constant need for a new active agent capable of reducing sebum secretion, said reduction being both visible and long-lasting, without causing a rebound effect, or any irritation of the skin, and without any loss of the radiance of the skin, and capable of preserving skin moisturization. There is also a need for a new active agent also capable of preventing and/or reducing the unaesthetic and/or unpleasant and/or uncomfortable manifestations of sebum secretion, such as the shiny, oily appearance of the skin and/or of the hair, pore visibility and/or blackhead formation and/or hyperkeratinization of the skin, in particular its thick appearance.

The present invention thus aims to meet these needs by virtue of a novel sebum secretion-inhibiting agent that is of use as an alternative to the active agents available on the market.

According to the present invention, it has in fact been discovered, particularly surprisingly and unexpectedly, that the Bixa orellana extract makes it possible to solve all these technical problems. The Bixa orellana extract according to the invention in fact makes it possible to very effectively prevent and/or reduce the secretion of sebum, in particular of total and/or neutral lipids by the sebocytes of the skin, including the scalp, in particular by inhibiting the activity of IGF-1 and/or by stimulating the synthesis of the IGBP3 protein (IGF1 binding protein 3), which is an inhibitor of IGF-1 activity. The extract according to the invention thus makes it possible to reduce sebocyte proliferation. This effect is both visible and long-lasting, without causing a rebound effect.

The extract according to the invention also has the advantage of preserving the moisturization and the radiance of the skin.

By directly inhibiting IGF1 activity and/or by stimulating the synthesis of the the IGBP3 protein (IGF1 binding protein 3), the Bixa orellana extract makes it possible to prevent and/or reduce the unaesthetic and/or unpleasant and/or uncomfortable manifestations of sebum secretion, such as the shiny and/or oily appearance of the skin and/or of the hair. By limiting keratinocyte proliferation and differentiation, the extract according to the invention makes it possible to reduce hyperkeratinization of the skin and thus the thick appearance of the skin. The extract according to the invention has the advantage of stimulating the synthesis and/or activity of type IV collagen by keratinocytes, therefore making it possible to tighten the pores of the skin and/or to reduce the number of dilated pores and/or to prevent skin pore dilation and and/or skin pore visibility and/or to reduce blackhead formation. In general, the skin grain is thus refined.

Moreover, the extract according to the invention makes it possible to reduce the secretory activity of sebocytes without completely stopping it, thus maintaining a minimum level of sebum secretion, essential for homeostasis of the skin and/or of the hair. In the long term, the extract according to the invention therefore does not dry out the skin.

By virtue of its complete action and these additional activities and devoid of the drawbacks of certain products on the cosmetics market, the extract according to the invention is therefore an excellent agent for inhibiting sebum secretion and the unaesthetic and/or unpleasant and/or uncomfortable manifestations thereof. It is thus particularly useful for the treatment and/or cosmetic care of normal, oily, very oily and/or mixed skin and/or normal and/or oily hair.

Likewise, it is thus particularly useful for the treatment and/or cosmetic care of Caucasian, Asian and/or African skin and/or Caucasian, Asian and/or African hair.

Bixa orellana is a shrub which belongs to the Bixa genus, belonging to the family Bixaceae. It is commonly known as “roucou” or lipstick tree. Extracts of this shrub have already been described in cosmetic applications for a whitening effect (JP08012563). Oily extracts of seeds have also been described as a photoprotective agent (FR2555447) and as a moisturizing agent (FR2798591).

None of the known properties of this extract nevertheless implied its effect for preventing and/or limiting sebum secretion and/or the unaesthetic manifestations of sebum secretion as discovered in the present invention.

The Bixa orellana extract has the advantage of acting on the sebocytes directly and with a long-term, long-lasting effect. In addition, the extract according to the invention prevents and/or reduces the skin imperfections associated with a high sebum production. It has in particular been observed that the extract according to the invention refines the grain of the skin by reducing pore visibility, in particular reducing pore size, and by making the grain more uniform and smoother.

Some of these properties of the Bixa orellana extract according to the invention will in particular be demonstrated in greater detail in the examples provided hereinafter.

A subject of the present invention is thus the cosmetic use of a Bixa orellana extract for preventing and/or reducing sebum secretion and/or for preventing and/or reducing the unaesthetic and/or unpleasant and/or uncomfortable manifestations linked to sebum secretion, in particular for preventing and/or reducing skin pore visibility and/or blackhead formation, and/or the shiny and/or oily appearance of the skin and/or of the hair and/or the thick appearance of the skin.

A subject of the present invention is also a Bixa orellana extract for use alone or in a pharmaceutical, in particular dermatological, composition, in the treatment and/or prevention of at least one pathological condition associated with sebum hyperproduction, in particular pathological hyperseborrhoea, seborrhoeic eczema and/or hypersecretion in newborn infants and/or any combination thereof.

A subject of the present invention is also the use according to the invention of a cosmetic or pharmaceutical, in particular dermatological, composition, comprising a Bixa orellana extract in combination with at least one agent chosen from sebum regulators, preferably chosen from sarcosine, zinc salicylate, zinc gluconate, azelaic acid, an Orthosiphon stamineus extract and/or derivatives thereof, and/or at least one agent with an additional property chosen from exfoliating, moisturizing or keratolytic agents, agents for stimulating fibronectin synthesis, for protecting fibroblast growth factor (FGF2), for stimulating fibroblast growth, an antibacterial agent, a sebum absorber, a comedolytic agent, a local antibiotic, and any mixture thereof.

A subject of the present invention is also the use of of such a cosmetic composition for preventing and/or reducing sebum secretion and/or for preventing and/or reducing the unaesthetic and/or unpleasant and/or uncomfortable manifestations linked to sebum secretion, in particular for preventing and/or reducing skin pore visibility and/or blackhead formation, and/or the shiny and/or oily appearance of the skin and/or of the hair and/or the thick appearance of the skin.

Finally, a subject of the present invention is a cosmetic care and/or treatment method comprising the topical, in particular daily, application, to at least one area of skin, advantageously of the scalp, of a Bixa orellana extract according to the invention or of a cosmetic composition according to the invention, for preventing and/or reducing sebum secretion and/or for preventing and/or reducing the unaesthetic and/or unpleasant and/or uncomfortable manifestations linked to sebum secretion, in particular for preventing and/or reducing skin pore visibility and/or blackhead formation, and/or the shiny and/or oily appearance of the skin and/or of the hair and/or the thick appearance of the skin.

A subject of the present invention is the cosmetic use of a Bixa orellana extract for preventing and/or reducing sebum secretion, preferentially in the form of a cosmetic composition.

A subject of the present invention is thus the use of a Bixa orellana extract for preventing and/or reducing sebum secretion and/or the unaesthetic and/or unpleasant and/or uncomfortable manifestations linked to sebum secretion.

The expression “preventing and/or reducing sebum secretion” is intended to mean decreasing or preventing the increase in the amount of sebum secreted by sebocytes in the skin, including the scalp, and advantageously the total and/or neutral lipids of sebum, in particular squalene, and/or decreasing or preventing sebocyte proliferation in the skin, including the scalp.

The amount of sebum secreted by the sebocytes can be measured in vitro by assaying the amount of cellular lipids produced in the sebocytes in culture with Nile Red staining according to the method described by Greenspan P et al. (Nile red: a selective fluorescent stain for intracellular lipid droplets. J Cell Biol. 100(3):965-73, (1985)) as described in Example 2.

The amount of sebum secreted can also be measured in vivo by clinical and instrumental analysis of sebum secretion samples on samples such as Sebutape™ patches according to the conventional method and/or by application of a sebumeter to the area of skin in question, preferentially the Sebumeter™ device sold by the company Courage+Khazaka Electronic GmbH, even more preferentially the SM815 model.

Advantageously, the extract according to the invention makes it possible to reduce by at least 5%, preferentially at least 10%, more preferentially 20%, the amount of sebum secreted compared with the control in the absence of extract, as is demonstrated in Example 2.

The sebocyte proliferation in the skin can be evaluated by counting the sebocytes in the presence of the extract according to the invention by staining DNA with Hoechst reagent according to the method described by Daxhelet et al (Spectrofluorometry of dyes with DNAs of different base composition and conformation; Analytical Biochemistry Volume 179, Issue 2, June 1989, Pages 401-403 Analytical Biochemistry 179:401-403 (1989) and presented in Example 2.

Advantageously, the extract according to the invention makes it possible to reduce by at least 5%, preferentially at least 10%, more preferentially at least 15%, the sebocyte proliferation compared with the control in the absence of extract preferentially as is demonstrated in Example 2.

The expression “unaesthetic and/or unpleasant and/or uncomfortable manifestations linked to sebum secretion” is intended to mean the shiny and/or oily appearance of the skin and/or of the hair, the skin pore visibility and/or blackhead formation, and/or the thick appearance of the skin. According to one particular embodiment of the invention, the unaesthetic and/or unpleasant and/or uncomfortable manifestations of sebum secretion are chosen from the shiny and/or oily appearance of the skin and/or of the hair, blackhead formation and the thick appearance of the skin.

For the purpose of the present invention, the expression “reducing skin pore visibility” is intended to mean tightening the pores of the skin, that is to say reducing the diameter of the pore opening, and/or the density and/or the area of the pores at the surface of the skin, and/or preventing skin pore dilation. The extract according to the invention therefore makes it possible to reduce the opening and/or the density of the pores at the surface of the skin.

The skin pore visibility may be demonstrated in vivo by an evaluation referred to as “scoring” by a dermatologist on a predefined area after application of a composition comprising the extract according to the invention, in particular as described in Example 5. It may also be demonstrated by an objective instrumental method by image analysis that makes it possible to extract and quantify specific parameters from high-resolution photographs, in cross-polarized configuration, of the face of volunteers taken before and after application of a composition comprising the extract according to the invention.

The density of the skin pores may also be measured in vivo by imaging, especially by the fringe projection technique, by measuring the parameter referred to as curvature.

In one preferred embodiment of the invention, the B. orellana extract according to the invention is in an effective amount for reducing skin pore visibility by at least 10%, preferentially by at least 20%, after 28 days of application of a cream comprising the B. orellana extract according to the invention, preferably the seed extract, more preferentially prepared under the conditions described in Example 1a), preferentially formulated in the form of a cosmetic composition as described in Example 5.

According to the invention, the expression “thick appearance of the skin” is intended to mean a hyperkeratinization of the skin. The decrease in hyperkeratinization of the skin can be measured by measuring the amount of IGF1-BP3 protein in the presence of the extract according to the invention on normal human keratinocytes in culture, in particular as presented in Example 3. Advantageously, the extract according to the invention is in an effective amount for increasing by at least 5%, preferentially at least 10%, more preferentially at least 20%, the amount of IGF1-BP3 protein synthesized in the presence of the extract according to the invention.

According to the invention, the expression “very oily skin” is intended to mean skin which has a lipid index, measured using a sebumeter applied directly to the area of skin to be evaluated, of greater than or equal to 150 micrograms of sebum per cm².

According to the invention, the expression “normal skin” is intended to mean skin which has a lipid index, measured using a sebumeter applied directly to the area of skin to be evaluated, of strictly less than 120 micrograms of sebum per cm², and preferentially greater than or equal to 100 micrograms of sebum per cm².

According to the invention, the expression “oily or seborrhoeic skin” is intended to mean skin which has a lipid index, measured using a sebumeter applied directly to the area of skin to be evaluated, ranging from 120 and 149 micrograms of sebum per cm². The expression “mixed skin” is intended to mean skin which has several profiles: oily, very oily, and/or normal as a function of the areas of the face under consideration, in particular oily skin on the T zone and normal-to-dry skin on the remainder of the face. According to the invention, the expression “normal hair” is intended to mean a scalp which has a lipid index, measured using a sebumeter applied directly to the area of the scalp to be evaluated, of less than or equal to 100 micrograms of sebum per cm².

According to the invention, the expression “oily hair” is intended to mean a scalp which has a lipid index, measured at measured using a sebumeter applied directly to the area of the scalp to be evaluated, of strictly greater than 100 micrograms of sebum per cm². Preferentially, the expression “sebumeter device” is intended to mean the Sebumeter™ device sold by the company Courage+Khazaka Electronic GmbH, even more preferentially the SM815 model.

For the purposes of the present invention, the expression “cosmetic use” is intended to mean a non-therapeutic, non-pharmaceutical use of the extract according to the invention, preferentially on healthy skin, in particular a healthy scalp, and/or healthy hair, in particular a use intended for all or part of the skin, preferentially of the scalp and/or for an area of skin, preferentially of scalp, referred to as “healthy”, particularly for an area of “healthy” skin and/or an area of “healthy” scalp.

For the purposes of the present invention, the expression “part of the skin, preferentially of the scalp, and/or area of skin, preferentially of the scalp, referred to as “healthy”” is intended to mean a part of the skin, preferentially of the scalp, and/or an area of skin, preferentially of the scalp, referred to as non-pathological by a dermatologist, that is to say which does not exhibit any infection, inflammation, scar, disease or ailment of the skin, such as folliculitis, candidiasis, psoriasis, ichthyosis, pathological conditions linked to melanogenesis, such as vitiligo, eczema, acne, actinic keratosis, carcinoma, melanoma, boil or dermatitis, in particular seborrhoeic dermatitis, alopecia or wounds or injuries. In particular, the expression “part of the skin, preferentially of the scalp” and/or “area of skin, preferentially of the scalp, referred to as “healthy”” is intended to mean a part of the skin, preferentially of the scalp, and/or an area of skin, preferentially of the scalp, referred to as “normal” by a physician, particularly a dermatologist, that is to say consisting of “normal” cells, that is to say non-pathological, more particularly non-cancerous, cells.

Unless indicated otherwise, the terms “normal” skin, “normal” cells, “normal” keratinocytes, “normal” sebocytes are intended to mean skin or cells that are not diseased.

The extract according to the invention is a Bixa orellana extract. It may be extracted from the entire plant or one or more parts of the plant, and in particular chosen from the root, the stalk, the bark, the flower, the seed, the germ and/or the leaf, and mixtures thereof. The extract according to the invention is preferentially a Bixa orellana seed extract.

The extract can be obtained by various extraction methods known to those skilled in the art, chosen from maceration, hot decoction, by milling including ultrasonic milling, using a mixer, or else the extract can be obtained by extraction in water under subcritical or supercritical conditions (carbon dioxide). Preferentially, the extraction is carried out by maceration.

The extraction may be carried out using dry or fresh matter, advantageously dry matter, in an amount of from 0.1% to 20% by weight, advantageously from 1% to 20%, very advantageously from 5% to 15%, more advantageously from 10% to 15%, and even more advantageously approximately 10% by weight relative to the total weight of the matter and of the extraction solvent.

The extraction may be carried out at a temperature ranging from 4° C. to 300° C., preferentially from 4° C. to 100° C. In one preferential embodiment of the invention, the extraction will be carried out at a temperature ranging from 60° C. to 90° C., preferentially from 70° C. to 85° C., more preferentially at a temperature of approximately 80° C.

According to one alternative embodiment, the extraction will be carried out at a temperature ranging from 4° C. to 20° C., more advantageously at ambient temperature, that is to say at approximately 20° C.

In another alternative embodiment of the invention, the extraction will be carried out in water under subcritical conditions, at a temperature ranging from 100° C. to 300° C., advantageously from 120° C. to 250° C., more advantageously at 120° C. The extraction can be carried out at a single given temperature or at successive increasing temperatures. In one advantageous embodiment of the invention, the extraction will be carried out at a single temperature of 120° C. In an alternative embodiment, it will be carried out according to a gradient of three increasing temperatures of between 100° C. and 200° C., such as 120° C., 140° C. then 160° C., or 110° C., 130° C. then 150° C., or else 120° C., 145° C. then 170° C.

The term extraction under “subcritical conditions” is intended to mean extraction in the presence of water, under conditions of temperature greater than 100° C. and pressure less than 221 bar, such that the water remains in the liquid state but has a viscosity and a surface tension lower than that of water at ambient temperature, increasing its dielectric constant.

Thus, the extraction pressure will be between 150 bar and 250 bar, preferentially between 200 and 221 bar, advantageously in a pressure extraction autoclave.

In general, the extraction can be carried out for a period of from 30 minutes to 24 hours, preferentially from 30 minutes to 12 hours, more preferentially for a period of from 1 hour to 5 hours, and more advantageously for a period of from 1 hour to 2 hours. Very advantageously, the extraction will be carried out for a period of one hour.

The extract according to the invention may be obtained by extraction in a solvent or solvent mixture, preferably a protic polar solvent, and advantageously in water, an alcohol, a glycol, a polyol, a water/alcohol, water/glycol or water/polyol mixture (such as water mixed with ethanol, glycerol and/or butylene glycol and/or other glycols such as xylitol and/or propanediol, etc.), from 99/1 to 1/99 (w/w), advantageously in water as sole solvent.

In one preferential embodiment, the extract is obtained by aqueous extraction. For the purposes of the present invention, the expression “extract obtained by aqueous extraction” is intended to mean any extract obtained by extraction with an aqueous solution containing more than 60% by weight, advantageously at least 70% by weight, in particular at least 80% by weight, more particularly at least 90% by weight, particularly at least 95% by weight, of water relative to the total weight of the aqueous solution, even more advantageously not containing glycol and in particular not containing alcohol, more particularly only containing water.

In yet another alternative embodiment, the extraction will be carried out in the presence of a C₆-C₁₆ dialkyl carbonate solvent with heptane. According to this embodiment, the extract according to the invention can be obtained by adding the part of Bixa orellana to be extracted, preferentially the seed (plant/solvent ratio=1/10) to the extraction solvent containing the dialkyl carbonate, more preferentially among dioctyl carbonate and diethylhexyl carbonate, with stirring for 2 hours at 80° C.

According to another embodiment, the extraction may be carried out with a solvent comprising coconut water. According to this embodiment, the extract according to the invention can be obtained by adding the part of Bixa orellana, preferentially the seed (plant/solvent ratio=1/10), with stirring, to the extraction solvent (a water/coconut water mixture), the pH of which has been adjusted to 3 or to 4, for 1 hour at 80° C. in a closed environment in order to avoid evaporation of the solvent. The extract is then cooled, centrifuged and filtered at 0.45 μm.

In another alternative embodiment of the invention, the extract may be obtained by extraction under supercritical conditions (CO₂) in the presence of a cosolvent. Advantageously, the cosolvent will be ethanol.

In another alternative embodiment of the invention, the extraction may be carried out in the presence of a non-ionic surfactant, preferentially chosen from lauryl glucoside sold under the name Plantacare® 1200UP by BASF or else caprylyl/capryl glucoside (Plantacare® 810 UP), preferentially caprylyl/capryl gluco side (Plantacare® 810 UP). The concentration by weight of the non-ionic surfactant may be between 0.5% and 5%, advantageously between 0.5 and 1%, more advantageously it will be 1% by weight relative to the total weight of the extract.

Advantageously according to the invention, the extract obtained after the extraction step will be filtered at a cut-off threshold of 0.45 μm. Additional decolorizing and/or deodorizing steps can be carried out on the extract at any stage of the extraction and according to the techniques known to those skilled in the art. In particular, the extract may be decolorized with activated carbon.

Moreover, the extract obtained after the extraction can then be concentrated by evaporation of the solvent or dried, for example by lyophilization or by spray-drying in the presence of a spray-drying support such as, for example, maltodextrin. The extract is then in powder form. According to one advantageous embodiment of the invention, the Bixa orellana extract obtained, preferentially of seeds, will be spray-dried in the presence of a concentration by weight of maltodextrin of between 20% and 90%, preferentially between 40 and 80%, more preferentially approximately 60%, relative to the total weight of the powder obtained.

Advantageously, the extract according to the invention is water-soluble.

The extract according to the invention is preferentially obtained according to one of the embodiments described below:

In a first embodiment of the invention, the extract is obtained by maceration, in water as solvent, of an amount of 10% by weight of milled seeds, relative to the weight of the solvent and of the material, for a period of one hour at a temperature of 80° C. The crude extract was centrifuged, decanted, then filtered and then spray-dried in the presence of maltodextrin, in a final amount of maltodextrin of 60% by weight relative to the total weight of the final extract, under the conditions described in Example 1a).

In a second embodiment of the invention, the extract is obtained by maceration, in water as solvent, of an amount of 10% by weight of milled seeds, relative to the total weight of the solvent and of the material, for a period of 2 hours at a temperature of 20° C. The crude extract was centrifuged, decanted and then filtered, under the conditions described in Example 1b).

In a 3^{rd} embodiment of the invention, the extract is obtained by maceration of an amount of 20% of milled seeds in water as solvent, for a period of 2 hours at a temperature of 20° C. The extract is then centrifuged, decanted and filtered, under the conditions described in Example 1c).

In a 4^{th} embodiment, the extract is obtained by maceration of an amount of 10% by weight of milled seeds relative to the total weight of the material and of the solvent, in an ethanol:water mixture (80:20; v/v), at a temperature of 80° C., for a period of 1 hour. The extract is then centrifuged, decanted, filtered and then spray-dried in the presence of maltodextrin, in a final amount of maltodextrin of 80% by weight relative to the total weight of the final extract, under the conditions described in Example 1d).

In a 5^{th} embodiment of the invention, the extract is obtained by maceration, in water as solvent, of an amount of 10% by weight of milled leaves, relative to the weight of the solvent and of the material, for a period of one hour at a temperature of 80° C. The crude extract was centrifuged, decanted, then filtered and then spray-dried in the presence of maltodextrin, in a final amount of maltodextrin of 60% by weight relative to the total weight of the final extract, under the conditions described in Example 1e).

According to the invention, the Bixa orellana extract according to the invention can be used alone in the form of an active ingredient and/or in a cosmetic composition, preferentially intended for topical application.

The term “topical application”, used herein, means applying the extract according to the present invention optionally in the form of an active ingredient and/or of a composition to the surface of the skin, including the scalp and/or the hair, in particular by direct application or by spraying.

The active ingredient and/or the cosmetic compositions containing the extract according to the invention are in particular intended for the cosmetic care and/or treatment of skin referred to as normal, oily, very oily and/or mixed and/or hair referred to as normal and/or oily, in particular oily, very oily and/or mixed skin and/or oily hair.

When it is used alone in the form of an active ingredient, the extract according to the invention is preferentially soluble in and/or diluted in a solvent, in particular a polar solvent, such as water, an alcohol, a polyol, a glycol, such as pentylene glycol and/or butylene glycol and/or hexylene glycol and/or caprylyl glycol, or a mixture thereof, preferentially an aqueous-glycolic or aqueous-alcoholic mixture, more preferentially containing a glycol chosen from hexylene glycol, caprylyl glycol and mixtures thereof. Advantageously, the extract according to the invention is soluble in and/or solubilized in an aqueous solution containing glycerol, advantageously in a solution containing at least 10% by weight of glycerol, preferentially at least 15%, and more advantageously 17.5% by weight of glycerol, relative to the total weight of the aqueous solution.

Alternatively, the extract obtained is diluted in and/or soluble in an aqueous solution containing caprylyl glycol, in particular containing between 0.01 and 5% by weight of caprylyl glycol, preferentially between 0.1 and 1% by weight of caprylyl glycol, relative to the total weight of the aqueous solution.

In another embodiment, the extract according to the invention can be incorporated in a cosmetic composition comprising at least one cosmetically acceptable excipient.

For the purposes of the present invention, the term “cosmetically acceptable” excipient is intended to mean a topically acceptable compound and/or solvent, that is to say which does not induce an undue allergic response on contact with the skin, including the human scalp, which is non-toxic, which is not unstable, or equivalents thereof.

For the purposes of the present invention, the term “cosmetic composition” is intended to mean a non-therapeutic composition, that is to say a composition intended for prevention and/or for care of the skin, including the scalp, referred to as “normal” by a dermatologist, that is to say non-pathological. The term “normal” skin or scalp is intended to mean here a healthy skin or scalp as defined above.

In one preferential embodiment of the invention, the extract according to the invention is present in the cosmetic composition in a content of between 1×10⁻⁴% to 10% by weight, preferentially from 1×10⁻⁴% to 5% by weight, more advantageously from 1×10⁻³% to 3% by weight, more preferentially from 0.001% and 0.1% by weight, relative to the total weight of the composition.

The cosmetic composition according to the invention may be in any of the galenical forms conventionally used for topical application to the skin, including the scalp, such as liquid or solid forms or even in the form of pressurized liquid. They may in particular be formulated in the form of an aqueous or oily solution, a cream or an aqueous gel or an oily gel, especially in a pot or tube, especially a shower gel, a shampoo, a conditioner, a milk, an oil, an emulsion, a hydrogel, a microemulsion or a nanoemulsion, especially oil-in-water or water-in-oil or multiple or silicone-based emulsion, a serum, a lotion, especially in a glass or plastic bottle or measuring bottle or an aerosol or spray, a vial, a liquid or solid soap, a paste, an ointment, a foam, a mask, a lacquer, a patch, an anhydrous product, which is preferably liquid, pasty or solid, for example in the form of a rod in particular in stick form, or in powder form. It may also be a makeup product or a makeup-removing product. In particular, the cosmetic composition is chosen from the group consisting of a serum, a lotion, a cream, a shampoo, a conditioner, an oil, a milk, an ointment, a paste, a foam, an emulsion, a hydrogel, a shower gel, a mask, a lacquer, a spray, and a wax; it is more preferentially a cream, a serum or a lotion.

Preferentially, the extract is particularly suitable for the formulation of a composition referred to as neutral and mild, for respecting the seborrhoea gland, in particular the skin, including the scalp.

As mentioned above, the Bixa orellana extract according to the present invention is preferentially used in the form of cosmetic or pharmaceutical, preferentially dermatological, compositions.

The compositions according to the invention may contain any appropriate solvent and/or any appropriate carrier and/or any appropriate excipient, optionally in combination with other compounds of interest.

As a result, for these compositions, the excipient contains, for example, at least one compound chosen from the group consisting of preservatives, emollients, emulsifiers, surfactants, moisturizers, thickeners, conditioning agents, matifying agents, stabilizers, antioxidants, texturing agents, sheen agents, film-forming agents, solubilizers, pigments, dyes, fragrances and sunscreens. These excipients are preferably chosen from the group consisting of amino acids and derivatives thereof, polyglycerols, esters, cellulose polymers and derivatives, lanolin derivatives, phospholipids, lactoferrins, lactoperoxidases, sucrose-based stabilizers, vitamins E and derivatives thereof, natural and synthetic waxes, plant oils, triglycerides, unsaponifiable matter, phyto sterols, plant esters, silicones and derivatives thereof, protein hydrolysates, jojoba oil and derivatives thereof, liposoluble/water-soluble esters, betaines, amine oxides, plant extracts, saccharose esters, titanium dioxides, glycines, and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, butylene glycol, natural tocopherols, glycerin, dihydroxycetyl sodium phosphate, isopropyl hydroxycetyl ether, glycol stearate, triisononanoin, octyl cocoate, polyacrylamide, isoparaffin, laureth-7, a carbomer, propylene glycol, glycerol, bisabolol, a dimethicone, sodium hydroxide, PEG-30 dipolyhydroxystearate, capric/caprylic triglycerides, cetearyl octanoate, dibutyl adipate, grape seed oil, jojoba oil, magnesium sulfate, EDTA, a cyclomethicone, xanthan gum, citric acid, sodium lauryl sulfate, mineral waxes and oils, isostearyl isostearate, propylene glycol dipelargonate, propylene glycol isostearate, PEG 8, beeswax, glycerides from hydrogenated palm kernel oil, glycerides from hydrogenated palm oil, lanolin oil, sesame oil, cetyl lactate, lanolin alcohol, castor oil, titanium dioxide, lactose, saccharose, low density polyethylene, an isotonic saline solution.

Many cosmetically active ingredients are known to those skilled in the art for improving the health and/or the physical appearance of the skin. Those skilled in the art know how to formulate cosmetic or dermatological compositions to obtain the best effects. Moreover, the compounds described in the present invention may have a synergistic effect when they are combined with each other. These combinations are also covered by the present invention. The CTFA Cosmetic Ingredient Handbook, Second Edition (1992) describes different cosmetic and pharmaceutical ingredients commonly used in the cosmetics and pharmaceutical industry, which are suitable in particular for topical use. Examples of these classes of ingredients comprise, without being limited thereto, the following compounds: abrasives, absorbents, compounds for aesthetic purposes such as fragrances, pigments, dyes, essential oils, astringents, etc. (for example: clove oil, menthol, camphor, eucalyptus oil, eugenol, menthyl lactate, witch hazel distillate), antiacne agents, anti-flocculant agents, antifoam agents, antimicrobial agents (for example: iodopropyl butyl carbamate), antioxidants, binders, biological additives, buffer agents, swelling agents, chelating agents, additives, biocidal agents, denaturing agents, thickeners, and vitamins, and derivatives or equivalents thereof, film-forming materials, polymers, opacifiers, pH regulators, reducing agents, depigmenting or lightening agents (for example: hydroquinone, kojic acid, ascorbic acid, magnesium ascorbyl phosphate, ascorbyl glucosamine), conditioning agents (for example: humectants).

Particularly advantageously, the Bixa orellana extract according to the invention may be used, optionally in a cosmetic or pharmaceutical, preferentially dermatological, composition, preferentially those described above, as sole active agent, in particular as sole sebum-regulating active agent, or in combination with one or more other active agents chosen from:

1) another cosmetic and/or dermatological sebum-regulating agent, preferentially sarcosine as sold by the applicant under the name MAT-XS™ Clinical, an Orthosiphon stamineus extract as sold by the applicant under the name MAT-XS™ Bright, zinc gluconate, zinc salicylate, azelaic acid, and/or derivatives thereof, and/or mixtures thereof, advantageously in combination with zinc gluconate.

and/or

2) an agent with additional properties, chosen from exfoliating and/or keratolytic agents, sebum-absorbing agents, agents which have an action on the microbiota, comedolytic agents and/or moisturizing agents.

In particular, mention may be made, as a preferential example:

of an exfoliating and/or keratolytic agent: alpha-hydroxy acids (AHAs), in particular salicylic acid, optionally in combination with acacia proteins, malic acid, optionally in combination with almond proteins, glycolic acid; lactic acid, and/or derivatives thereof; and/or mixtures thereof;

of a sebum-absorbing agent: a talc and/or an absorbent polymer;

of an agent which has an action on the microbiota, in particular the skin microbiota:

the antibacterial agents described in patent application FR2863893, and in particular a Peumus boldus extract, such an extract being in particular sold by the applicant under the name Betapur™, and/or a local antibiotic agent, in particular erythromycin and/or clindamycin phosphate,

agents which balance the microbial flora, such as a mixture of pullulan, sodium alginate and sodium hyaluronate sold by the applicant under the name PatcH20™, in particular combined with serine, trehalose and urea and described in patent application WO2014027163A2, and/or a Saccharomyces cerevisiae extract for increasing the cutaneous and/or mucosal commensal microbial flora sold by the applicant under the name Relipidium™;

of a comedolytic agent: retinoic acid or a derivative thereof, such as isotretinoin, adapalene and/or 13-cis-retinoic acid and benzoyl peroxide;

of moisturizing agents: one or more agents which promote moisturization, such as a polysaccharide extracted from Cassia angustifolia seeds and sold under the name Hyalurosmooth™ by the applicant, or an agent chosen from one of the combinations containing pullulan, sodium hyaluronate and sodium alginate sold under the name PatcH2O™ by the applicant, or else one or more of the natural moisturizing factor compounds (Natural Moisturizing Factor) or a natural honey extract sold by the applicant under the name Melhydran™, and/or a compound of the family of glucosyl glycerides, in particular hexosyl glyceride, a Litchi chinensis pericarp extract under the name Litchiderm™ by the applicant.

Preferentially, the cosmetic composition containing the extract according to the invention will contain another sebum-regulating agent chosen from sarcosine and an Orthosiphon stamineus extract, zinc gluconate and mixtures thereof, a moisturizing agent chosen from Cassia angustifolia seed extract, hexosyl glyceride, a mixture containing pullulan, sodium hyaluronate and sodium alginate, and mixtures thereof. Advantageously, the composition containing the extract according to the invention will also contain an agent which acts on the bacterial flora, chosen from a Peumus boldus extract, a mixture of pullulan, sodium alginate and sodium hyaluronate, a Saccharomyces cerevisiae extract, and mixtures thereof.

The cosmetic compositions containing the extract according to the invention may contain the conventional active agents of cosmetic compositions, in particular chosen from:

an agent for stimulating fibronectin synthesis, in particular a corn extract, such an extract being in particular sold by the applicant under the name Deliner™;

an agent for protecting the fibroblast growth factor (FGF2) of the extracellular matrix against its degradation and/or its denaturing, in particular a Hibiscus abelmoschusextract as described in the patent application in the name of the applicant filed under the number FR0654316 and/or an agent for stimulating fibroblast growth, for example an extract of fermented soya containing peptides, known under the name Phytokine™ sold by the applicant, and also described in patent application EP1119344 B1 (Laboratoires Expanscience), and preferentially a combination of these two extracts;

an agent for stimulating laminin synthesis, in particular a biotechnology-modified malt extract, such an extract being in particular sold by the applicant under the name Basaline™;

an agent for stimulating the expression and/or activity of hyaluronan synthase 2 (HAS2), such as the plant extracts described in patent application FR2 893 252 A1, and in particular an aqueous extract of Galanga (Alpinia galanga);

an agent for stimulating lysyl oxidase like (LOXL) synthesis, such as those described in patent application FR2855968, and in particular a dill extract;

one or more anti-pollution agents such as an Argania spinosa leaf extract sold under the name Arganyl™ or a Moringa oleifera seed extract sold under the name Purisoft™ by the applicant, or else an Eperua falcata root extract sold under the name Eperuline™;

an agent for stimulating intracellular ATP synthesis, in particular an extract of the alga Laminaria digitata;

an agent with an overall anti-ageing action, especially an anti-pigment spot agent, in particular niacinamide or vitamin B3;

and any mixture thereof.

According to the present invention, the use of the Bixa orellana extract according to the invention is particularly advantageous in that it allows a complete, efficient and long-lasting sebum-regulating action on any type of skin and/or hair, in particular Caucasian or African or Asian skin and/or Caucasian, African or Asian hair, in particular Caucasian or Asian skin and/or Caucasian or Asian hair, more particularly Asian skin and/or Asian hair.

Preferentially, the Bixa orellana extract according to the invention, preferentially in the form of a cosmetic composition according to the invention, is applied to at least one area of the body where the sebum secretion is uncomfortable and/or unaesthetic and/or unpleasant, this area or these areas preferentially being a surface of the body chosen from the skin of the face, including the forehead, the cheeks, the nose, the temples, the T zone (forehead, nose and chin), the external auditory canal and/or the chin, the scalp, the neck, the back, the shoulders, the forearms, the chest, the hands and/or the bust.

A subject of the present invention is also a cosmetic care method in which the Bixa orellana extract according to the invention is applied to at least one part of the body, preferentially a surface chosen from the skin of the face, including the forehead, the cheeks, the nose, the temples, the T zone (forehead, nose and chin), the external auditory canal and/or the chin, the scalp, the neck, the back, the shoulders, the forearms, the chest, the hands and/or the bust, preferentially for preventing and/or reducing sebum secretion and/or for preventing and/or reducing the unaesthetic and/or uncomfortable and/or unpleasant effects of sebum secretion.

Advantageously, the Bixa orellana extract according to the invention, preferentially in the form of a cosmetic composition according to the invention, is used by regular topical application, preferentially at least once a day, advantageously twice a day, for at least 10 days, preferentially for 20 days, and more preferentially for at least 40 days. Preferentially, the cosmetic composition is applied to the skin without rinsing, by massaging.

Advantageously, a subject of the invention is also a cosmetic treatment method for preventing and/or reducing the sebum secretion of an individual who requires same/who desires same, comprising the following steps:

a) the identification on the individual of an area of skin for which it is desired to reduce sebum secretion and/or the unaesthetic and/or unpleasant and/or uncomfortable manifestations linked to sebum secretion, and

b) the topical application, to this area of skin, of a cosmetic composition containing the Bixa orellana extract according to the invention in an effective amount for preventing and/or reducing sebum secretion on this area of skin, namely in an extract content of between 1×10⁻⁴% to 10% by weight, preferentially from 1×10⁻⁴% to 5% by weight, more advantageously from 1×10⁻³% to 3% by weight, more preferentially from 0.001% and 0.1% by weight, relative to the total weight of the composition.

The Bixa orellana extract according to the invention can be used for the treatment and/or prevention of a pathological condition associated with sebum hyperproduction, in particular pathological hyperseborrhoea, seborrhoeic eczema and/or hypersecretion in newborn infants and/or any combination thereof.

Preferentially, the Bixa orellana extract according to the invention, preferentially in the form of a pharmaceutical composition according to the invention, is applied to at least one area of the body where the sebum secretion is excessive, is induced and/or is associated with at least one pathological condition, in particular to at least one area of the body exhibiting pathological hyperseborrhoea, this area or these areas preferentially being a surface of the body chosen from the skin of the face, including the forehead, the cheeks, the nose, the temples, the T zone (forehead, nose and chin), the external auditory canal and/or the chin, the scalp, the neck, the back, the shoulders, the forearms, the chest, the hands and/or the bust.

In one preferential embodiment of the invention, the extract according to the invention is present in the pharmaceutical composition in a content of between 1×10⁻⁴% to 10% by weight, preferentially from 1×10⁻⁴% to 5% by weight, more advantageously from 1×10⁻³% to 3% by weight, more preferentially from 0.001% and 0.1% by weight, relative to the total weight of the composition.

Other aims, features and advantages of the invention will emerge clearly to those skilled in the art on reading the explanatory description, which makes reference to examples that are given purely as illustrations and shall not in any way limit the scope of the invention.

The examples form an integral part of the present invention, and any feature appearing to be novel relative to any prior art whatsoever, from the description taken in its entirety, including the examples, forms an integral part of the invention in its function and in its general nature.

Thus, each example has a general scope.

Moreover, in the examples, all the percentages are given on a weight basis, unless otherwise indicated, the temperature is expressed in degrees Celsius, unless otherwise indicated, and the pressure is atmospheric pressure, unless otherwise indicated.

### EXAMPLE

### Example 1: Different Methods for Preparing the Bixa orellana Extract according to the Invention

Example 1a): An amount of 10% (w/w relative to the total mixture of water and seeds) of milled Bixa orellana seeds was macerated in water as solvent for one hour at a temperature of 80° C. The crude extract was centrifuged, decanted, then filtered and then spray-dried in the presence of maltodextrin, in a final amount of maltodextrin of 60% by weight relative to the total weight of the final extract.

Example 1b): An amount of 10% by weight of milled seeds (relative to the total weight of water and seeds) was macerated in water for 2 hours at a temperature of 20° C. The crude extract was centrifuged, decanted and then filtered.

Example 1c): An amount of 20% by weight of milled seeds (relative to the total weight of water and seeds) was macerated in water for 2 hours at a temperature of 20° C. The extract was then centrifuged, decanted and filtered.

Example 1d): An amount of 10% by weight of milled seeds (relative to the total weight of the seeds and of the solvent) was macerated in a solvent consisting of an ethanol:water mixture (80:20; v/v), at a temperature of 80° C. for a period of 1 hour. The extract was then centrifuged, decanted, filtered and then spray-dried in the presence of maltodextrin, in a final amount of maltodextrin of 80% by weight relative to the total weight of the final extract.

Example 1e): An amount of 10% by weight of milled leaves (relative to the total weight of water and leaves) was macerated in water for one hour at a temperature of 80° C. The crude extract was centrifuged, decanted, then filtered and then spray-dried in the presence of maltodextrin, in a final amount of maltodextrin of 60% by weight relative to the total weight of the final extract.

### Example 2: Properties of a Bixa orellana Extract according to the Invention on Sebum Secretion

### Method

Human sebocytes were seeded at 2500 cells/centimetre² in a complete culture medium (DMEM/HAM F12 with foetal calf serum (FCS) at 10% (w/w in the medium) and incubated for 5 days at 37° C., CO₂ at 5% with a relative humidity of more than 95%. Then the growth culture medium was replaced with a standard culture medium (DMEM) containing human serum (HS) at 1% or IGF1 at 100 ng/ml. The Bixa orellana extract obtained in Example 1a) as obtained before the step of mixing with the maltodextrin was incubated for 5 days at 37° C. at 0.02% or 0.05% by weight/weight relative to the mixture of medium and the extract. The cells are rinsed in a PBS buffer (phosphate buffered saline) and fixed with a formaldehyde solution. The number of sebocytes is determined by DNA staining with Hoechst reagent and the fluorescence is recorded at 465 nm (excitation at 356 nm) while the amount of cellular lipids is determined with Nile Red staining (11). The fluorescence of total lipids is measured at a wavelength of 625 nm (excitation at 520 nm) while the fluorescence of neutral lipids is measured at a wavelength of 530 nm (excitation at 475 nm). The results are expressed as percentage relative to the control without the Bixa orellana extract (the mean basal expression with HS at 1% or IGF1 at 100 ng/mL) and presented as a mean +/−standard deviation (SD). The statistics were evaluated with the SigmaPlot™ software.

### Results

### 1. Activation with 1% HS

### Asian Sebocytes, 25 Years Old

N = 3

| | | HS at 1% | |
|---|---|---|---|
| | without HS | Control | Extract at 0.02% |
| Number of cells (DNA) | 61 +/− 27 | 100 +/− 6 | 83 +/− 4 |
| Total lipids | 50 +/− 14 | 100 +/− 4 | 84 +/− 7 |
| | | | p < 0.001 |
| Neutral lipids | 74 +/− 24 | 100 +/− 5 | 76 +/− 24 |
| | | | p < 0.001 |

The Bixa orellana extract according to the invention significantly reduced sebocyte proliferation and the synthesis of total lipids in sebocytes treated with 1% HS.

### 2. Activation with IGF-1 100 ng/ml

### 2.1. Caucasian Sebocytes, 25 Years Old

| | | IGF at 100 ng/ml | |
|---|---|---|---|
| | without IGF | Control | Extract 0.05% |
| Number of cells | 81 +/− 6 | 100 +/− 8 | 48 +/− 3 |
| (DNA) | | | p < 0.001 |
| Total lipids | 77 +/− 11 | 100 +/− 7 | 33 +/− 3 |
| | | | p < 0.001 |
| Neutral lipids | 94 +/− 1 | 100 +/− 3 | 86 +/− 2 |
| | | | p < 0.01 |

The Bixa orellana extract according to the invention, at a concentration of 0.05%, significantly reduced sebocyte proliferation and the synthesis of total and neutral lipids in sebocytes treated with IGF-1. Similar results were obtained at 0.02% of extract according to the invention.

### 2.2. Asian Sebocytes, 25 Years Old

| | | IGF at 100 ng/ml | |
|---|---|---|---|
| | without IGF | Control | Extract 0.05% |
| Number of cells | 89 +/− 5 | 100 +/− 8 | 69 +/− 9 |
| (DNA) | | | p < 0.05 |
| Total lipids | 88 +/− 10 | 100 +/− 11 | 53 +/− 11 |
| | | | p < 0.01 |

The Bixa orellana extract according to the invention, at a concentration of 0.05%, significantly reduces sebocyte proliferation and the synthesis of total lipids in sebocytes treated with IGF-1.

### Example 3: Induction of the IGH-Binding Protein: IGFBP3 Protein

The BP3 protein is dedicated to blocking the binding of IGF-1 to its respective receptor IGF1R; it will therefore block the IGF1-IGF1R pathway and therefore obstruct keratinocyte hyperproliferation and differentiation. By activating this protein, the Bixa orellana extract slows down the hyperkeratinization observed in oily skin. The Bixa orellana extract according to the invention increases the synthesis of the IGFBP3 protein.

### Materials and Methods

Normal human keratinocytes were cultured up to 70% confluence, then treated for 24 h with IGF-1 at 100 ng/ml or the Bixa orellana extract obtained according to Example 1a) before mixing with maltodextrin and spray-drying at 0.1% or 0.05% (w/w in the total medium) for 24 h. The cells are lysed for assaying the DNA, and the supernatant is used for the quantification of the IGFBP3 protein by ELISA according to the protocol of the supplier of the ELISA kit (LSBio Human IGFBP3 ELISA Kit ref LS-F24538 lot 103180).

### Results

The results are related to non-treated cells. A statistical t-test test is carried out, and the means are compared.

| | Mean | deviation | Statistic |
|---|---|---|---|
| Non-treated | 100 | 19.3937 | |
| IGF-1 | 128.195 | 25.6252 | NS |
| extract 0.1% | 147.769 | 25.3724 | P < 0.01 |
| extract 0.05% | 174.824 | 24.9337 | P < 0.001 |

The Bixa orellana extract according to the invention increased the amount of IGFBP3 protein.

### Example 4: Increase in Collagen IV Synthesis by the Extract according to the Invention

### Method

“Normal” human keratinocytes originating from the breast of a healthy 24-year-old donor were cultured in a defined medium (KSFM) for a period of 48 hours until confluence (100%) in the presence of 3 different final concentrations of the B. orellana extract prepared according to Example 1a), before the mixing and spray-drying, then the culture medium is removed. The cell layer obtained is then lysed with an ammonium hydroxide solution. A part of the lysate was used to assay the DNA. The collagen IV was assayed with an anti-collagen IV antibody (Acris, R1041) diluted to 1/2500 in a PBS buffer solution containing bovine serum albumin (BSA). After a period of 60 minutes, the secondary antibody (PerkinElmer, AD0105) diluted to 1/25000 is applied for a period of 1 hour in darkness. The fluorescence was measured (ENVision, PerkinElmer).

### Results

The fluorescence results were standardized relative to the fluorescence obtained with the same cell medium in the absence of the B. orellana extract (Control) and were related to the percentage of DNA obtained under each condition. The results presented correspond to the mean of the assays. (SD: Standard deviation).

| | Mean | Standard deviation | statistic |
|---|---|---|---|
| Non-treated | 100.00 | 7.53 | |
| extract 0.025% | 144.08 | 8.67 | p < 0.001 |
| Extract 0.05% | 157.83 | 7.77 | p < 0.001 |
| Extract 0.1% | 159.83 | 14.64 | p < 0.001 |

The Bixa orellana extract according to the invention increases collagen IV synthesis in the keratinocytes and thus makes it possible to tighten the pores of the skin.

### Example 5: Clinical Study of a Bixa orellana Extract according to the Invention on Sebum Secretion

### Method

A clinical study was carried out double blind, in a half-face test, one part with the placebo and one part with the Bixa orellana extract according to the invention at 0.25% as obtained in Example 1a) and formulated in the composition described below (Formula according to the invention).

The study was carried out on 35 Asian women aged from 20 to 45 years old, with a lipid index, measured using a sebumeter (Sebumeter™ SM 815), ≥150 micrograms of sebum per cm² (very oily skin) and including a maximum of 10 volunteers with a lipid index, measured using a sebumeter (Sebumeter™ TM SM 815), ranging from 120 to 149 micrograms of sebum per cm² (oily skin). The treatment was applied twice a day for 56 days.

| | | | Formula |
|---|---|---|---|
| | | Placebo | according to the |
| | | Formula | invention |
| Brand name | INCI | (% w/w) | (% w/w) |
| Eumulgin ® | Sodium stearoyl | 0.5 | 0.5 |
| SG | glutamate | | |
| 10% Citric acid | Citric acid, aqua | 0.7 | 0.65 |
| solution | | | |
| Cosmedia ® | Sodium polyacrylate | 0.7 | 0.7 |
| SP | | | |
| Cutina ® PES | Pentaerythrityl | 1 | 1 |
| | distearate | | |
| Glycerine 99-5 | Glycerin | 2 | 2 |
| Elestab ™ 388 | Propylene glycol, | 2.5 | 2.5 |
| | phenoxyethanol, | | |
| | chlorphenesin, | | |
| | methylparaben | | |
| Emulgade ™ | Sucrose polystearate, | 3 | 3 |
| Sucro Plus | cetyl palmitate | | |
| Miritol ™ 318 | Caprylic/capric | 3 | 3 |
| | triglyceride | | |
| Cetiol ® C-5-C | Coco-caprilate/caprate | 3 | 3 |
| Cetiol ® CC | Caprylyl carbonate | 3 | 3 |
| Extract according | Bixa orellana | 0 | 0.25 |
| to the invention | | | |
| according to Ex | | | |
| 1a) | | | |
| Water | Aqua | qs | qs |

The compositions were prepared by mixing according to the methods known by those skilled in the art.

Samples were taken using the Sebutape® patch and made it possible to measure the activity of the sebaceous glands by measuring the amount of sebum before (D0) and after treatment at 28 and 56 days. The size of the pores on the cheeks, evaluated at the start between 2 and 4, was evaluated by a clinical calculation carried out by a trained referent individual. The moisturization of the skin was measured using the corneometry method. A questionnaire was filled in by the volunteers at 28 and 56 days.

### Results

At 28 days of treatment with the Bixa orellana extract according to the invention at 0.25%, the number of spots (secreted sebum) significantly decreases by 22% versus D0, and significantly by 14% versus placebo.

At 56 days of treatment with the Bixa orellana extract according to the invention at 0.25%, the number of spots (secreted sebum) significantly decreases by 37% versus D0, and significantly by 25% versus placebo.

At 28 days with the Bixa orellana extract according to the invention at 0.25%, the area of the spots significantly decreases by 46% versus D0, and significantly by 31% versus placebo.

At 56 days of treatment with the Bixa orellana extract according to the invention at 0.25%, the number of spots (secreted sebum) significantly decreases by 62% versus D0, and significantly by 36% versus placebo.

At 28 days of treatment with the Bixa orellana extract according to the invention at 0.25%, the size of the pores decreases visually and significantly by 3% versus D0. At 56 days of treatment with the Bixa orellana extract according to the invention at 0.25%, the size of the pores significantly decreases by 9% versus D0, and by 3% versus placebo.

After 28 and 56 days of treatment, the Bixa orellana extract according to the invention at 0.25% significantly increases the corneometer value by 3% versus D0.

### Example 6: Cosmetic Composition according to the Invention

The extract used is that obtained in Example 1a) (after mixing with maltodextrin and spray-drying) in powder form.

### Example 6a): Tonic Care for Refining the Grain of the Skin

| | | Amount (% by |
|---|---|---|
| Phase | Name | total weight) |
| A | Water | 87.65 |
| A | Glycerin | 4.00 |
| A | Disodium EDTA | 0.05 |
| A | Preservative | qs |
| A | Polysorbate 20 | 1.00 |
| A | Poloxamer 184 | 2.00 |
| A | Zinc gluconate | 0.05 |
| B | water | 3 |
| B | Bixa orellana extract according to Example 1a) | 0.25 |
| B | pH adjuster (citric acid) | qs |
| C | PEG-7 glyceryl cocoate | 0.5 |
| H | Fragrance | 0.1 |

The tonic care is prepared by the usual methods in the field well known to those skilled in the art, by mixing the 3 phases and by adjusting the composition to a pH of 5.1.

### Example 6b): Matifying Cream

| | | Amount (% by |
|---|---|---|
| Phase | Name | total weight) |
| A | Sucrose polystearate, cetyl palmitate | 3.00 |
| A | Pentaerythrityl distearate | 1.00 |
| A | Caprylic/capric triglyceryl | 3.00 |
| A | Coco-caprylate/caprate | 3.00 |
| A | Dicaprylyl carbonate | 3.00 |
| A | Sodium polyacrylate | 0.70 |
| B | water | 80.90 |
| B | glycerin | 2.00 |
| B | Sodium stearoyl glutamate | 0.50 |
| B | preservative | qs |
| C | Bixa orellana extract according to Example 1a) | 0.25 |
| C | Water | 2.00 |
| D | Fragrance | qs |
| E | pH adjuster (citric acid) | qs |

The cream is prepared by the usual methods in the field well known to those skilled in the art, by mixing phases A and B preheated to 75° C., and then by adding phases C and D while mixing and while adjusting the composition with phase E to a pH of 6.2 and to a viscosity of 15000 mPas (measured with a Brookfield instrument (RVT; 23° C., spindle TC; 20 rev per min)).

### Example 6c): Shampoo

| | | Amount (% by |
|---|---|---|
| Phase | Name | total weight) |
| A | Water | 60.3 |
| A | Xanthan gum | 1.2 |
| B | Decyl glucoside | 14 |
| B | Dicaprylyl ether, decyl glucoside, glyceryl oleate | 5 |
| B | Sodium cocoyl glutamate | 12 |
| B | Coco-glucoside, glyceryl oleate | 2 |
| B | Glycerin | 3 |
| B | Preservative | qs |
| C | pH adjuster (citric acid) | qs |
| D | Fragrance | 0.5 |
| D | Bixa orellana extract according to Example 2a) | 0.01-10 |

The shampoo is prepared by the usual methods in the field well known to those skilled in the art, by mixing the 4 phases and by adjusting the composition to a pH of 5.2 and to a viscosity of 2200 mPas (measured with a Brookfield instrument (RVT; 23° C., spindle 5; 50 rev per min)).

1.-17. (canceled)

18. A cosmetic method for preventing and/or reducing sebum secretion and/or for preventing and/or reducing the unaesthetic and/or unpleasant and/or uncomfortable manifestations linked to sebum secretion of an individual who requires or desires same, comprising the following steps:
a) identifying on the individual an area of skin and/or hair for which it is desired to reduce sebum secretion and/or the unaesthetic and/or unpleasant and/or uncomfortable manifestations linked to sebum secretion, and
b) applying to this area of skin and/or hair a Bixa orellana extract in an effective amount for preventing and/or reducing sebum secretion and/or the unaesthetic and/or unpleasant and/or uncomfortable manifestations linked to sebum secretion on this area of skin and/or hair.

19. The cosmetic method according to claim 18, wherein the unaesthetic and/or unpleasant and/or uncomfortable manifestations of sebum secretion are skin pore visibility and/or blackhead formation and/or the shiny and/or oily appearance of the skin and/or of the hair and/or the thick appearance of the skin.

20. The cosmetic method according to claim 18, wherein the Bixa orellana extract is a seed extract.

21. The cosmetic method according to claim 18, wherein the Bixa orellana extract is obtained by extraction in a protic polar solvent.

22. The cosmetic method according to claim 21, wherein the Bixa orellana extract is spray-dried in the presence of a concentration by weight of maltodextrin of between 20% and 90%, relative to the total weight of the powder obtained.

23. The cosmetic method according to claim 18, wherein the Bixa orellana extract is in the form of a cosmetic composition also comprising a cosmetically acceptable excipient.

24. The cosmetic method according to claim 23, wherein the cosmetic composition is intended for topical application to the skin and/or the hair.

25. The cosmetic method according to claim 24, wherein the Bixa orellana extract is present in the cosmetic composition in a content of between 1×10⁻⁴% to 10% by weight, relative to the total weight of the composition.

26. The cosmetic method according to claim 24, in which the skin is normal, oily, very oily and/or mixed and/or the hair is normal and/or oily.

27. The cosmetic method according to claim 24, in which the skin is Caucasian or African or Asian and/or the hair is Caucasian, African or Asian.

28. The cosmetic method according to claim 23, wherein the cosmetic composition is chosen from the group consisting of a serum, a lotion, a cream, a shampoo, a conditioner, an oil, a milk, an ointment, a paste, a foam, an emulsion, a hydrogel, a shower gel, a mask, a lacquer, a spray, and a wax.

29. (canceled)

30. (canceled)

31. The cosmetic care method according to claim 23, wherein the area of skin for which it is desired to reduce sebum secretion and/or the unaesthetic and/or unpleasant and/or uncomfortable manifestations linked to sebum secreation is a surface of the body chosen from the skin of the face, the cheeks, the nose, the temples, the T zone (forehead, nose and chin), the external auditory canal and/or the chin, the scalp, the neck, the back, the shoulders, the forearms, the chest, the hands and/or the bust.

32. A method for the treatment and/or prevention of a pathological condition associated with sebum hyperproduction chosen from pathological hyperseborrhoea and seborrhoeic eczema and/or any combination thereof of an individual who requires or desires the same, comprising the following steps:
a) identifying on the individual of an area of akin for which it is desired to treat and/or prevent a pathological condition associated with sebum hyperproduction chosen from pathological hyperseborrhoea and seborrheic eczema and/or any combination thereof, and
b) applying to this area of skin a Bix orellana extract in an effective amount for treating and/or preventing a pathological condition associated with sebum hyperproduction chosen from pathological hyperseborrhoea and seborrheic eczema and/or any combination thereof on this area of skin.

33. The method according to claim 32, wherein the extract is a seed extract.

34. The method according to claim 32, wherein the extract is present in a pharmaceutical composition in a content of between 1×10⁻⁴% to 10% by weight relative to the total weight of the composition.

35. The method according to claim 32, wherein the extract is present in a pharmaceutical composition in a content of between 1×10⁻⁴% to 5% by weight, relative to the total weight of the composition.

36. The cosmetic method according to claim 21, wherein the Bixa orellana extract is spray-dried in the presence of a concentration by weight of maltodextrin of between 40 and 80%, relative to the total weight of the powder obtained.

37. The cosmetic method according to claim 21, wherein the Bixa orellana extract is spray-dried in the presence of a concentration by weight of maltodextrin of 60%, relative to the total weight of the powder obtained.

38. The cosmetic method according to claim 21, wherein the Bixa orellana extract is an extract obtained by aqueous extraction.

39. The method according to claim 32, wherein the Bixa orellana extract is an extract obtained by aqueous extraction.
